(19) United States
(12) Patent Application Publication
(10) Pub. No.: US 2020/0312456 A1
(43) Pub. Date: Oct. 1, 2020

(54) MACHINE-LEARNING BASED MEDICAL ANALYSIS SYSTEM AND METHOD THEREFOR

(71) Applicants: Orly YADID-PECHT, Calgary (CA); Jie YANG, Alberta (CA)

(72) Inventors: Orly YADID-PECHT, Calgary (CA); Jie YANG, Alberta (CA)

(21) Appl. No.: 16/830,921

(22) Filed: Mar. 26, 2020

Related U.S. Application Data
(60) Provisional application No. 62/824,093, filed on Mar. 26, 2019.

(51) Int. Cl. G16H 50/20; G16H 50/30; G16H 10/60; G16H 30/00; G16H 80/00; G06N 3/04; G06N 3/08; G06N 20/00

(52) U.S. Cl. CPC G16H 50/20; G16H 50/30; G16H 10/60; G06N 20/00; G16H 80/00; G06N 3/0454; G06N 3/08; G16H 30/00

(57) ABSTRACT

System and method for generating a recommendation for an individual, based on input data related to that individual. The input data is passed to a data-abstraction module which produces preprocessed data, which is then passed to an expert module that comprises a plurality of expert units and an expert manager. Each expert unit is configured to analyze the preprocessed data with respect to a specific predefined medical area and to generate at least one treatment option related to that specific predefined medical area. The expert manager is configured to receive such treatment options from the plurality of expert units, and to generate final treatment suggestion(s) based on the recommendations. As would be understood, the expert manager can consider different factors of importance for each individual. The final treatment suggestion(s) is then presented to at least one user. In some embodiments, the various modules comprise machine-learning modules.

### RELATED APPLICATIONS

This application is a non-provisional patent application that claims the benefit of U.S. Provisional Application No. 62/824,093 filed on Mar. 26, 2019.

### FIELD OF THE DISCLOSURE

The present disclosure relates generally to a medical analysis system and method, and in particular to a medical analysis system using machine learning and a method therefor.

### BACKGROUND

Wearable devices with health monitoring functions are known. With the development of wearable technologies, more and more personal data may be recorded by various wearable devices. For example, sleep patterns, real-time heart rate, and daily walking-distances may be recorded by suitable wristband devices. The accumulated time-sequence data is helpful for analyzing users' health conditions and give treatment suggestions thereto.

In recent years, most medical records are digitized thereby allowing them to be easily aggregated. The aggregation of digitized medical-records provides doctors an opportunity to combine various medical records and obtain a more comprehensive understanding of patient's medical histories, compared to doctors' historical practice which had to view and analyze the medical records separately due to the difficulty in aggregating many types of medical-records.

For example, while a patient's daily sleep-pattern alone may indicate a sleep problem, the sleep-pattern may also be an indication of a different problem such as a coronary heart disease due to an underlying relationship between one's long-term sleep pattern and coronary heart disease. However, the daily data and medical records of many individuals together give rise to a large amount of information that may be beyond the knowledge and processing ability of human experts.

In the past few years, the advancement of machine-learning algorithms made it possible to find the underlying correlations of various types of data and bring intelligent diagnosis systems to the medical society. In prior art, such intelligent medical diagnosis systems are mostly in the field of medical imaging domain.

For example, the academic paper entitled “Gene selection for cancer classification using support vector machines,” by Isabelle Guyon, Jason Weston, Stephen Barnhill, Vladimir Vapnik, and published in Machine Learning, January 2002, Volume 46, Issue 1-3, pp 389-422, teaches a support vector machine used for gene selection for cancer classification.

The academic paper entitled “Breast cancer diagnosis using least square support vector machine,” by K. Polat and S. Guneş, and published in Digital Signal Processing, vol. 17, no. 4, pp. 694-701, 2007, teaches a least-square support machine for breast cancer diagnosis.

Bayesian methods have been used in medical diagnosis (see academic paper entitled “inductive and bayesian learning in medical diagnosis,” by I. Kononenko, and published in Applied Artificial Intelligence, vol. 7, no. 4, pp. 317-337, 1993), and has shown a good performance in dealing with incomplete statistics (see academic paper entitled “Constructing Bayesian networks for medical diagnosis from incomplete and partially correct statistics,” by D. Nikovski, and published in IEEE Transactions on Knowledge and Data Engineering, vol. 12, no. 4, pp. 509-516, 2000). The academic paper entitled “Construction of a Bayesian network for mammographic diagnosis of breast cancer,” by C. Kahn, L. Roberts, K. Shaffer and P. Haddawy, and published in Computers in Biology and Medicine, vol. 27, no. 1, pp. 19-29, 1997, teaches that Bayesian methods may also be used in mammographic diagnosis.

However, the above-described methods have not gained many uses in real-world diagnosis because of their unstable performance and unsatisfactory accuracy. In recent years, the development of deep neural networks (DNNs) has greatly boosted many fields such as object detection and recognition. Some deep learning system/algorithms have achieved better-than-human accuracy; see academic paper entitled “Deep Residual Learning for Image Recognition,” by K. He, X. Zhang, S. Ren and J. Sun, and published in 2016 IEEE Conference on Computer Vision and Pattern Recognition (CVPR), Las Vegas, N V, 2016, pp. 770-778.

The development of machine learning has made intelligent medical diagnosis systems practical. In 2015 Lipton presented a Long Short-Term Memory (LSTM) based network to help medical diagnosis; see “Learning to diagnose with LSTM recurrent neural networks,” by Zachary Chase Lipton, David C. Kale, Charles Elkan, Randall C. Wetzel, published in ArXiv, 2015, and accessible at https://arxiv.org/abs/1511.03677. It is also reported that Tencent AI Lab (whose website is accessible at https://ai.tencent.com/ailab/index.html) uses machine-learning techniques in their systems for assisting doctors to read magnetic resonance imaging (MRI) and X-ray images for detecting lung cancers.

Kermany et al. teach the use of transfer learning for diagnosing retina diseases; see academic paper entitled “Identifying Medical Diagnoses and Treatable Diseases by Image-Based Deep Learning,” by D. Kermany, M. Goldbaum, W. Cai, C. Valentim, H. Liang, S. Baxter, A. McKeown, G. Yang, X. Wu, F. Yan, J. Dong, M. Prasadha, J. Pei, M. Ting, J. Zhu, C. Li, S. Hewett, J. Dong, I. Ziyar, A. Shi, R. Zhang, L. Zheng, R. Hou, W. Shi, X. Fu, Y. Duan, V. Huu, C. Wen, E. Zhang, C. Zhang, O. Li, X. Wang, M. Singer, X. Sun, J. Xu, A. Tafreshi, M. Lewis, H. Xia and K. Zhang, and published in Cell, vol. 172, no. 5, pp. 1122-1131.e9, 2018.

Recently, a work from Google that can help predict patents' life expectation was reported in the scientific journal, Nature; see academic paper entitled “Scalable and accurate deep learning with electronic health records,” by A. Rajkomar, E. Oren, K. Chen, A. Dai, N. Hajaj, M. Hardt, P. Liu, X. Liu, J. Marcus, M. Sun, P. Sundberg, H. Yee, K. Zhang, Y. Zhang, G. Flores, G. Duggan, J. Irvine, Q. Le, K. Litsch, A. Mossin, J. Tansuwan, D. Wang, J. Wexler, J. Wilson, D. Ludwig, S. Volchenboum, K. Chou, M. Pearson, S. Madabushi, N. Shah, A. Butte, M. Howell, C. Cui, G. Corrado and J. Dean, and published in npj Digital Medicine, vol. 1, no. 1, 2018.

The above-described works have shown great performance in diagnosis and some of them exhibit better prediction accuracy over human doctors. In 2017, a deep neural network was used to classify skin cancer as accurately as board-certified dermatologists; see academic paper entitled “Dermatologist-level classification of skin cancer with deep neural networks,” by A. Esteva, B. Kuprel, R. Novoa, J. Ko, S. Swetter, H. Blau and S. Thrun, and published in Nature, vol. 542, no. 7639, pp. 115-118, 2017.

In 2018, researchers reported a deep-learning model that can recognize elevated cardiovascular-disease risk from photographs of the retina fundus; see academic paper entitled “Prediction of cardiovascular risk factors from retinal fundus photographs via deep learning,” by R. Poplin, A. Varadarajan, K. Blumer, Y. Liu, M. McConnell, G. Corrado, L. Peng and D. Webster, and published in Nature Biomedical Engineering, vol. 2, no. 3, pp. 158-164, 2018.

### SUMMARY

This invention provides a system and method for generating a recommendation for an individual, based on input data related to that individual. The input data is passed to a data-abstraction module which produces preprocessed data, which is then passed to an expert module that comprises a plurality of expert units and an expert manager. Each expert unit is configured to analyze the preprocessed data with respect to a specific predefined medical area and to generate at least one treatment option related to that specific predefined medical area. The expert manager is configured to receive such treatment options from the plurality of expert units, and to generate final treatment suggestion(s) based on the recommendations. As would be understood, the expert manager can consider different factors of importance for each individual. The final treatment suggestion(s) is then presented to at least one user. In some embodiments, the various modules comprise machine-learning modules.

A first aspect of the invention provides system for generating a recommendation for an individual, said system comprising: a data-abstraction module for receiving input data related to said individual and for producing preprocessed data based on said input data; and an expert module for analyzing said preprocessed data, said expert module comprising: a plurality of expert units, wherein each of said plurality of expert units is configured to analyze said preprocessed data with respect to a specific predefined medical area, and each of said plurality of expert units is also configured to generate at least one treatment option related to said specific predefined medical area; and an expert manager, wherein said expert manager is configured to receive outputs from said plurality of expert units and to generate at least one final treatment suggestion based on said outputs, wherein said at least one final treatment suggestion is presented to at least one user.

A second aspect of the invention provides a method for generating a recommendation for an individual, said method comprising: receiving input data related to said individual; producing preprocessed data based on said input data; analyzing said preprocessed data with respect to a plurality of specific predefined medical areas, to thereby generate at least one treatment option related to each of said plurality of specific predefined medical areas; generating at least one final treatment suggestion based on outputs of said analyzing step; and presenting said at least one final treatment suggestion to at least one user.

A third aspect of the invention provides non-transitory computer-readable media having encoded thereon computer-readable and computer-executable instructions that, when executed, implement a method for generating a recommendation for an individual, said method comprising: receiving input data related to said individual; producing preprocessed data based on said input data; analyzing said preprocessed data with respect to a plurality of specific predefined medical areas, to thereby generate at least one treatment option related to each of said plurality of specific predefined medical areas; generating at least one final treatment suggestion based on said recommendations; and presenting said at least one final treatment suggestion to at least one user.

### BRIEF DESCRIPTION OF THE DRAWINGS

FIG. 1 is a schematic diagram of a medical analysis system, according to some embodiments of the present disclosure;

FIG. 2 is a schematic diagram showing a simplified hardware structure of a computing device of the medical analysis system shown in FIG. 1;

FIG. 3 a schematic diagram showing a simplified software architecture of a computing device of the medical analysis system shown in FIG. 1;

FIG. 4 is a block diagram showing a functional architecture of the medical analysis system shown in FIG. 1;

FIG. 5 is a block diagram showing a functional architecture of a convolutional neural network of the medical analysis system shown in FIG. 1 for preprocessing medical images;

FIG. 6 is a block diagram showing a functional architecture of a Long Short-Term Memory (LSTM) unit of the medical analysis system shown in FIG. 1 for preprocessing medical records and activity data, the LSTM unit comprising a plurality of LSTM cells;

FIG. 7 is a block diagram showing a functional architecture of a LSTM cell shown in

FIG. 6;

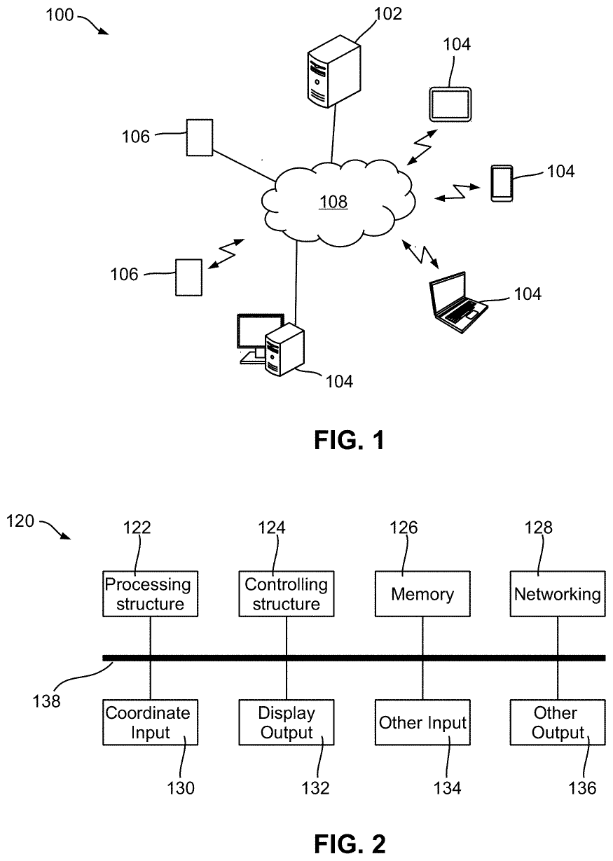

FIG. 8 is a block diagram showing a functional architecture of an expert of the medical analysis system shown in FIG. 1 for providing treatment suggestions;

FIG. 9 shows an example of a datasheet comprising a plurality of data points of various data types obtained at various time instants, wherein some data points therein are missing; and

FIG. 10 is a flowchart outlining a method according to one embodiment of the invention.

### DETAILED DESCRIPTION

### System Architecture Overview

The embodiments disclosed herein relate to a system and method for generating at least one recommendation for individuals, based on data related to those individuals. More specifically, the embodiments disclosed herein relate to a system and method for gathering human-activity data such as sleep pattern, daily walking distance, blood-pressure records, medical records (e.g., magnetic resonance imaging (MRI) images, medical notes, and medicine history), and/or the like, analyzing users' health status, and providing corresponding treatment suggestions. The human-activity data and medical records are formatted and computed through a deep neural network (DNN) architecture to provide reliable treatment-options for users to choose. The system and method uses machine learning in analyzing medical-image data and time-sequence data.

As will be described in more detail below, the collected input data is passed to a data-abstraction module which preprocesses the input data. The resulting preprocessed data is then passed to an expert module, which comprises a plurality of expert units and an expert manager. Each expert unit is configured to analyze the preprocessed data with respect to a specific predefined medical area (e.g., “diet control”) and to generate a treatment recommendation related to that specific predefined medical area. The expert manager is configured to receive such treatment recommendations from the plurality of expert units, and to generate a final treatment suggestion based on the recommendations. As would be understood, the expert manager may consider different factors of importance for each individual. The final treatment suggestion is then presented to at least one user, who may be the individual, a medical practitioner, and/or a caregiver for the individual.

Turning now to FIG. 1, a medical analysis system is shown and is generally identified using reference numeral 100. The medical analysis system 100 collects and analyzes medical images, medical records, daily activity data, and the like to predict users' health status over time and provides variety of treatment suggestions for the users to choose. The input data may further comprise data related to a history of the individual, to a family history of the individual, and other health- and well-being related data.

As would be understood by the person skilled in the art, the data may be input to the system by various means including automatic data input using various devices (such as wearable devices) that automatically collect data. Additionally, the data may be input manually by various individuals such as individuals to whom the data relates, medical personnel, caregivers, or any other suitable person. For instance, a doctor or nurse might upload medical imaging scans for an individual, while the individual might provide data related to their sleeping patterns.

Additionally, it should be understood that not all individuals will have the same data types input to the system. For instance, it is likely that far more medical images will be input to the system in the case of a patient recovering from emergency surgery than for an individual having a standard medical checkup at the office of their primary care physician. However, as would be clear, it is preferable to input as much initial data as possible.

The input data may have many different forms. For instance, the input data may comprise at least one of: text data; image data; video data; medical imaging data; numerical data; unidimensional data; or multi-dimensional data.

As shown in FIG. 1, in one implementation, the medical analysis system 100 comprises one or more server computers 102, a plurality of client computing devices 104, and one or more data-input sources 106 for inputting medical images, medical records, daily activity data, and the like. The one or more server computers 102, the client computing devices 104, and the data-inputs 106 are functionally interconnected by a network 108, such as the Internet, a local area network (LAN), a wide area network (WAN), a metropolitan area network (MAN), and/or the like, via suitable wired and wireless networking connections.

The server computer 102 executes one or more server programs. Depending on implementation, the server computer 102 may be a server-computing device, and/or a general purpose computing device acting as a server computer while also being used by a user.

Each client computing device 104 executes one or more client application programs (or so-called “apps”) for users to use. The client computing devices 104 may be portable computing devices such as laptop computers, tablets, smartphones, Personal Digital Assistants (PDAs), and/or the like. However, those skilled in the art will appreciate that one or more client computing devices 104 may be non-portable computing devices such as desktop computers in some embodiments.

Generally, the computing devices 102 and 104 have a similar hardware structure such as a hardware structure 120 shown in FIG. 2. As shown, the computing device 102/104 comprises a processing structure 122, a controlling structure 124, memory or storage 126, a networking interface 128, coordinate input 130, display output 132, and other input and output modules 134 and 136, all functionally interconnected by a system bus 138.

The processing structure 122 may be one or more single-core or multiple-core computing processors such as INTEL® microprocessors (INTEL is a registered trademark of Intel Corp., Santa Clara, Calif., USA), AMD® microprocessors (AMD is a registered trademark of Advanced Micro Devices Inc., Sunnyvale, Calif., USA), ARM® microprocessors (ARM is a registered trademark of Arm Ltd., Cambridge, UK) manufactured by a variety of manufactures such as Qualcomm of San Diego, Calif., USA, under the ARM® architecture, or the like.

The controlling structure 124 comprises one or more controlling circuits, such as graphic controllers, input/output chipsets and the like, for coordinating operations of various hardware components and modules of the computing device 102/104.

The memory 126 comprises a plurality of memory units accessible by the processing structure 122 and the controlling structure 124 for reading and/or storing data, including input data and data generated by the processing structure 122 and the controlling structure 124. The memory 126 may be volatile and/or non-volatile, non-removable or removable memory such as RAM, ROM, EEPROM, solid-state memory, hard disks, CD, DVD, flash memory, or the like. In use, the memory 126 is generally divided to a plurality of portions for different use purposes. For example, a portion of the memory 126 (denoted as storage memory herein) may be used for long-term data storing, for example, for storing files or databases. Another portion of the memory 126 may be used as the system memory for storing data during processing (denoted as working memory herein).

The networking interface 128 comprises one or more networking modules for connecting to other computing devices or networks through the network 108 by using suitable wired or wireless communication technologies such as Ethernet, WI-FI®, (WI-FI is a registered trademark of Wi-Fi Alliance, Austin, Tex., USA), BLUETOOTH® (BLUETOOTH is a registered trademark of Bluetooth Sig Inc., Kirkland, Wash., USA), ZIGBEE® (ZIGBEE is a registered trademark of ZigBee Alliance Corp., San Ramon, Calif., USA), 3G, 4G and/or 5G wireless mobile telecommunications technologies, and/or the like. In some embodiments, parallel ports, serial ports, USB connections, optical connections, or the like may also be used for connecting other computing devices or networks although they are usually considered as input/output interfaces for connecting input/output devices.

The display output 132 comprises one or more display modules for displaying images, such as monitors, LCD displays, LED displays, projectors, and the like. The display output 132 may be a physically integrated part of the computing device 102/104 (for example, the display of a laptop computer or tablet), or may be a display device physically separate from but functionally coupled to other components of the computing device 102/104 (for example, the monitor of a desktop computer).

The coordinate input 130 comprises one or more input modules for one or more users to input coordinate data, such as touch-sensitive screen, touch-sensitive whiteboard, trackball, computer mouse, touchpad, or other human interface devices (HID). The coordinate input 130 may be a physically integrated part of the computing device 102/104 (for example, the touch-pad of a laptop computer or the touch-sensitive screen of a tablet), or may be a display device physically separate from, but functionally coupled to, other components of the computing device 102/104 (for example, a computer mouse). The coordinate input 130, in some implementations, may be integrated with the display output 132 to form a touch-sensitive screen or touch-sensitive whiteboard.

The computing device 102/104 may also comprise other input 134 such as keyboards, microphones, scanners, cameras, Global Positioning System (GPS) component, and/or the like. The computing device 102/104 may further comprise other output 136 such as speakers, printers and/or the like.

The system bus 138 interconnects various components 122 to 136 enabling them to transmit and receive data and control signals to and from each other.

It should be clear that the medical analysis system, according to one aspect of the present invention, may comprise one or more computing devices that performs that analysis. Data can be input into the computing device either automatically or manually and the software modules within the computing device can execute methods and processes that embody the other aspects of the present invention. These methods and processes, as well as the software modules, are described in greater detail below.

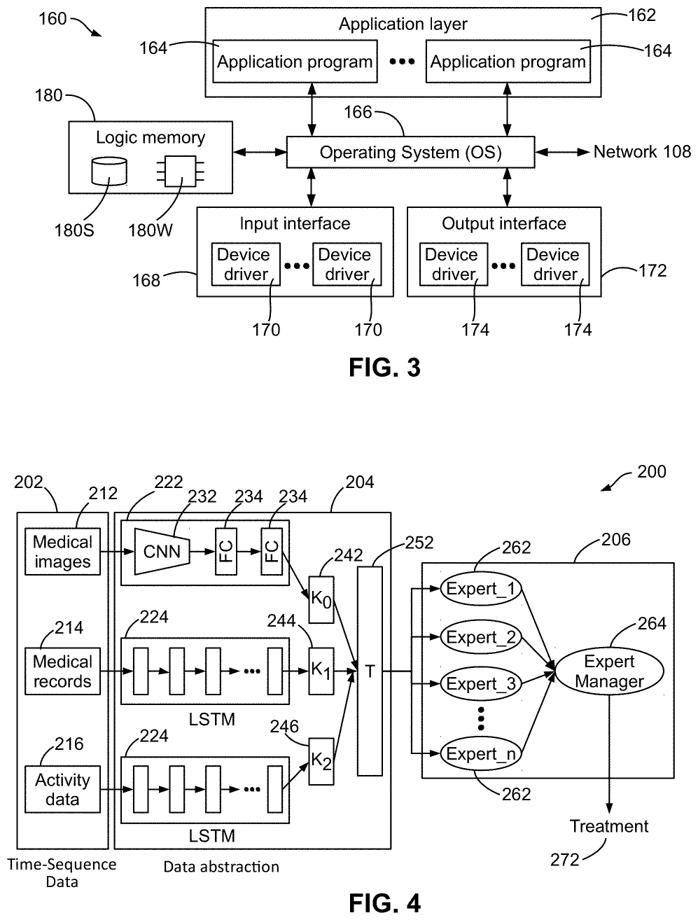

FIG. 3 shows a simplified software architecture 160 of the computing device 102 or 104. The software architecture 160 comprises an application layer 162, an operating system 166, an input interface 168, an output interface 172, and a logic memory 180. The application layer 162 comprises one or more application programs 164 executed by or run by the processing structure 122 for performing various tasks. The operating system 166 manages various hardware components of the computing device 102 or 104 via the input interface 168 and the output interface 172, manages the logic memory 180, and manages and supports the application programs 164. The operating system 166 is also in communication with other computing devices (not shown) via the network 108 to allow application programs 164 to communicate with those running on other computing devices. As those skilled in the art will appreciate, the operating system 166 may be any suitable operating system such as MICROSOFT® WINDOWS® (MCROSOFT and WINDOWS are registered trademarks of the Microsoft Corp., Redmond, Wash., USA), APPLE® OS X, APPLE® iOS (APPLE is a registered trademark of Apple Inc., Cupertino, Calif., USA), Linux, ANDROID® (ANDRIOD is a registered trademark of Google Inc., Mountain View, Calif., USA), or the like. The computing devices 102 and 104 of the medical analysis system 100 may all have the same operating system, or may have different operating systems.

The input interface 168 comprises one or more input device drivers 170 for communicating with respective input devices including the coordinate input 130. The output interface 172 comprises one or more output device drivers 174 managed by the operating system 166 for communicating with respective output devices including the display output 132. Input data received from the input devices via the input interface 168 is sent to the application layer 162 and is processed by one or more application programs 164. The output generated by the application programs 164 is sent to respective output devices via the output interface 172.

The logical memory 180 is a logical mapping of the physical memory 126 for facilitating the application programs 164 to access. In this embodiment, the logical memory 180 comprises a storage memory area (180S) that may be mapped to a non-volatile physical memory such as hard disks, solid state disks, flash drives, and the like, generally for long-term data storage therein. The logical memory 180 also comprises a working memory area (180W) that is generally mapped to high-speed, and in some implementations volatile, physical memory such as RAM, generally for application programs 164 to temporarily store data during program execution. For example, an application program 164 may load data from the storage memory area 180S into the working memory area 180W and may store data generated during its execution into the working memory area 180W. The application program 164 may also store some data into the storage memory area 180S as required or in response to a user's command.

In a server computer 102, the application layer 162 generally comprises one or more server-side application programs 164 which provide server functions for managing network communication with client computing devices 104 and facilitating collaboration between the server computer 102 and the client computing devices 104. Herein, the term “server” may refer to a server computer 102 from a hardware point of view or a logical server from a software point of view, depending on the context.

With above-described hardware structure of system 100, various data of one or more users may be collected by the data-input sources 106 and sent to the one or more servers 102 for medical analysis. Users may use their client computing devices 104 to query the analytical results and corresponding treatment suggestions and/or advices.

FIG. 4 is a block diagram showing a functional architecture 200 of the medical analysis system 100, according to some embodiments of this disclosure. As shown, the medical analysis system 100 comprises a neural network architecture having input time-sequence data 202 collected from various data-input sources 106, a data-abstraction module 204, and an expert module 206.

In these embodiments, the input time-sequence data 202 various medical data such as medical images 212, medical records 214, and daily-activity data 216, which may be stored and presented in different forms. For example, the medical images 212 may be image data containing pixel information while the medical records 214 and daily-activity data 216 may be sequenced text-data.

To extract the information of different formats, the medical analysis system 100 uses a convolutional neural network (CNN) unit 222 and one or more long-short term memory (LSTM) units 224 to process the image and sequence text, respectively. The CNN unit 222 comprises a CNN layer 232 and a plurality of fully-connected (FC) layers 234 and generates a K₀ dimensional vector 242 that represents a high-level abstract of the original image.

The LSTM units 224 generate K₁ and K₂ dimensional vectors 244 and 246 for the medical records 214 and activity data 216, respectively. In these embodiments, each LSTM unit 224 comprises an encoder network (described in more detail later) for generating a fixed-length vector representation from input data although traditional LSTM architecture uses an encoder-decoder architecture for encoding the input data to a fixed-length vector which is then decoded to sentences, text, and/or the like.

The generated vectors 242, 244, and 246 are concatenated to form a long vector T (also identified using reference numeral 252) that represents all information at the current time instant. Then vector T is used as input to the expert module 206 for analysis and for providing treatment suggestion 272 in a specific predefined medical area/area of expertise. As shown, the expert module 206 comprises a plurality of expert units 262 such as Expert_1, Expert_2, . . . , Expert_n, and an expert manager 264. Each expert unit 262 is an individual decoder network that decodes the vector T to a specific kind of treatment. For example, Expert_1 may be specialized in diet control, Expert_2 may be specialized in treatments with Chinese medicine, and Expert_3 may be specialized in using surgery techniques for treating diseases. Each expert unit 262 may conduct an analysis and provides a suggestion or option in its specific area. As should be understood, the treatment options may be related to medical procedures, activities, diet, exercise, or any other health-related or expertise-related option.

The expert manager 264 gathers all treatment options from the expert units 262 and provides at least one final treatment suggestion 272, which is a weighted combination of treatment suggestions provided by expert units 262. Thus, the architecture 200 is different from other machine-learning based medical diagnosis systems because of the use of multiple expert units 262. In some embodiments, the expert manager 264 may gather the advice from all or some expert units 262 and further use an additional neural network to process the gathered advice before outputting the final treatment suggestion 272.

Additionally, in some embodiments, the original input data and/or the preprocessed data (i.e., the vector T) may also be passed to the expert manager 264. The expert manager 264 may use such data as a reference point when generating the final treatment suggestion 272. That is, portions of the initial data may be treated as weighing factors by the expert manager 264 when generating the final treatment suggestion 272. In such embodiments, further, the individual's preferences may be taken into account, to increase potential likelihood of compliance with the treatment.

For instance, the treatment recommendation for an individual who indicates a strong preference for a certain kind of treatment (e.g., a preference for “holistic” health measures) may be different than for an individual with a similar condition who indicates no faith in such a treatment. In this example, because of the preference for holistic health measures, such holistic measures or holistic measure related options could be given a higher weighting when generating the final treatment suggestion. Because of this, the final treatment suggestion for the individual who prefers holistic health measures would be more likely to include such holistic health measures.

Additional factors in the input data and/or the preprocessed data (e.g., pre-existing conditions) may also receive higher weightings for some individuals than others, or cause certain recommendations from specific predefined medical areas to be more highly weighted than others. For instance, a recommendation related to oncology could be given a higher weight for an individual who is known to have cancer than for an individual who is not known to have cancer.

Further, as would be understood, the at least one final treatment suggestion 272 may be presented to the individual directly, to medical practitioners and/or caregivers, and/or to any other suitable person. The recipient of such final treatment suggestion 272 may depend on who initially input the data, on the nature of the suggestion itself (e.g., if the recommendation is a specific surgery, it would be preferable to inform the intended surgeon, rather than only informing the individual), and on many other factors. In some embodiments, the final treatment suggestion 272 may be presented to any user of a client computing device 104, for instance by on-screen notifications.

### System Architecture Detail

The system 100, or more specifically the neural-network architecture 200 thereof, aims to maximize the probability of the correct health-condition description given the medical data 202 using the following formula:

θ*=arg max Σ log p(S|I;θ), (1)

where θ is the parameter of the neural-network architecture, θ* is an approximation of θ obtained through the optimization of Equation (1), I is the time-sequence data 202, and S=[S₀, S₁, S₂, . . . , S_{N}] is the correct health-condition description of a user. In these embodiments, θ, θ*, I, and S are vectors.

Here, the chain rule is applied to model the joint probability over S₀, S₁, S₂, . . . , S_{N}, where Sᵢ(0≤i≤N) represents a health-condition description, and

log p(S|I;θ)=log p(Sₜ|I,S₀,S₁ . . . ,Sₜ₋₁).

During neural-network training, (S, I) is an example, and the sum of the log probabilities may be optimized using stochastic gradient decent.

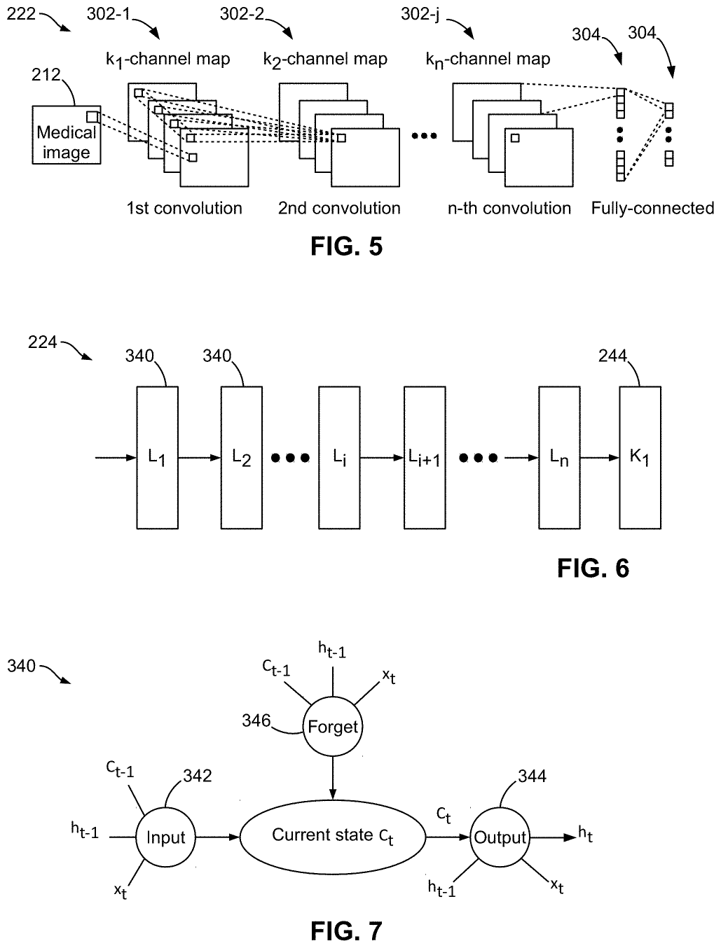

The overall architecture 200 shown in FIG. 4 contains preprocessing of medical images 212, medical records 214, and activity data 216. FIG. 5 is a block diagram showing a functional architecture of the CNN unit 222 for preprocessing medical images 212.

As shown, the CNN unit 222 comprises n convolutional layers 302-1, 302-2, . . . , 302-n (collectively identified using reference numeral 302) coupled in series. Each layer 302-i (i=1, 2, . . . , n) is a convolutional layer with k different kernels generating kᵢ channel maps. In these embodiments, kᵢ>kⱼ if i<j and thus the decreased number of kernels reduce the number of channel maps, and consequently reduce the computational cost.

Each convolutional layer may be described as:

F(X)=max(0,W*X+B), (3)

where W and B represent the kernel parameters and bias of convolution operations, respectively, and max( ) represents the Rectified Linear Unit (ReLU). After processing the medical image 212 using n convolutional layers, the output of the n-th convolutional layer is further processed by two fully-connected layers 304 which further reduce the dimension of the data to obtain the K₀ dimensional vector 242 representing all information contained in the medical image. ReLU is an activation function defined as the positive part of its argument, i.e.,

f(x)=max(0,x). (4)

Convolutional layers 302 and fully-connected layers 304 have connections between layers but do not have connections there within, which limits their capability of processing time-sequence data.

FIG. 6 is a block diagram showing a functional architecture of the LSTM unit 224 for preprocessing medical records 214 and activity data 216. As shown, the LSTM unit 224 comprises n identical LSTM layers 340 and receives the medical data or activity data records 214 and 216 as the input thereof.

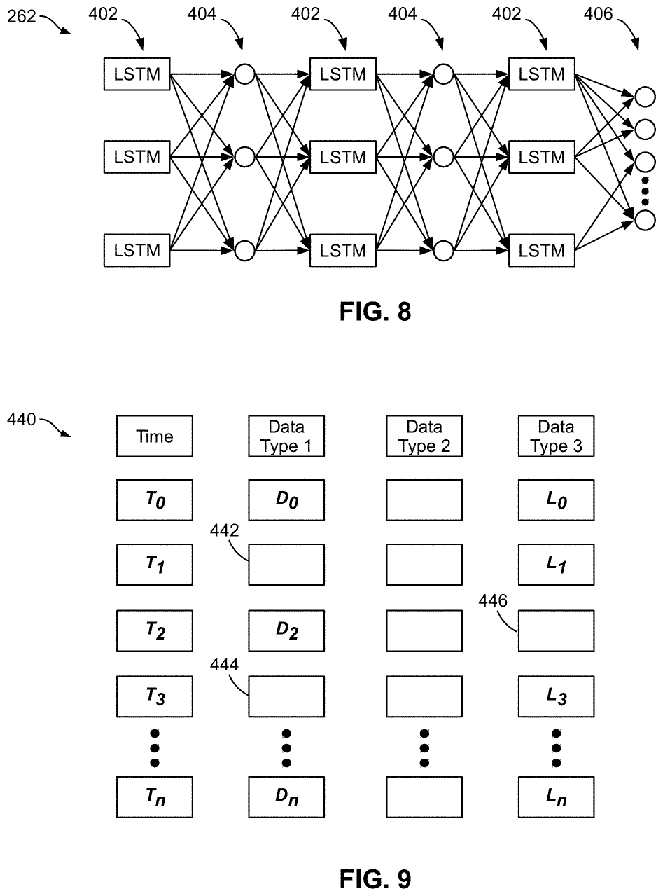

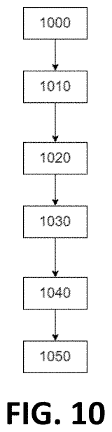

Each LSTM cell 340 is a type of recurrent neural network and may “remember” or “forget” contents thereof. An exemplary architecture of the LSTM cell 340 is shown in FIG. 7, which contains three different “gates”, namely, an input gate 342, an output gate 344, and a forget gate 346. The gates 342, 344, and 346 may selectively allow the incoming information affect the state Cₜ of the neural network.

The forget gate 346 and the input gate 342 are the core components to keep long-term memory wherein the forget gate 346 may erase some unwanted information in the memory. As shown, the forget gate 346 uses the current input xₜ, the previous status Cₜ₋₁, and the previous output hₜ₋₁ to decide which part of the memory should be erased:

fₜ=σ(W_{f}·[hₜ₋₁,xₜ]+b_{f}), (5)

where σ represent sigmoid operation, and W_{f} and b_{f} are the kernel and the bias, respectively. Memory status will add new memory through the input gate 342 after the unwanted memory is erased:

iₜ=σ(Wᵢ·[hₜ₋₁,xₜ]+bᵢ), (6)

\tilde{C}ₜ=tan h(W_{C}·[hₜ₋₁,xₜ]+b_{C}). (7)

In above equations, \tilde{C}ₜ is a candidate vector and iₜ decides which elements within vector \tilde{C}ₜ should be kept. The updated status of the cell is:

Cₜ=fₜ*Cₜ₋₁+iₜ*\tilde{C}ₜ. (8)

The output signal is then computed as:

σ(Wₒ[hₜ₋₁,xₜ]+bₒ)*tan h(Cₜ), (9)

where Wₒ and bₒ are the kernel parameter and bias, respectively.

FIG. 8 is a block diagram showing a functional architecture of the expert unit 262 for providing treatment suggestions. As shown, the expert unit 262 comprises three LSTM layers 402, two feed-forward layers 404, and a softmax layer 406 which provides treatment suggestions and/or predictions.

### Data Format

Medical and activity data 214 and 216 gathered from various data-input sources 106 may have different formats. More importantly, medical and activity data 214 and 216 may have multiple missing data-points which may make the neural-network training impossible. Therefore, it is important to convert all input data into a standard format and supplement missing data-points.

FIG. 9 shows an example of a datasheet 440 comprising a plurality of data points forming a plurality of data sequences of various data types such as Data Type 1, Data Type 2, and Data Type 3, obtained at various time instants T₀, T₁, . . . , Tₙ. In some embodiments, each data point may be in the form of a file, and these files may be denoted as data-sequence files.

Each data type has one or more missing data-points represented therein by “blank” blocks (i.e., blocks with no text therein). For example, data points 442 and 444 of Data Type 1 at time instants T₁ and T₃ are missing, all data points of Data Type 2 are missing, and the data point 446 of Data Type 3 at time instant T₂ is missing.

Various suitable methods may be used for supplementing missing data points. For example, the missing data-points 442 and 444 of Data Type 1 in time instants T₁ and T₃ may be supplemented by using a suitable interpolation method such as a bilinear interpolation method. Alternatively, the missing data-points 442 and 444 may be supplemented by copying the nearest neighboring data points such as data points D₀ and D₂ thereto.

If all data points such as those of Data Type 2 are missing, one may use an average value of that data type to supplement the missing data-points. Such an average value may be predetermined based on the data type thereof and the user's known characteristics. For example, if all data points of the data type of heart rate are missing, and the user is male and is in his mid-60s, then one may supplement the missing heart-rate data-points by using an average heart rate of male people in their mid-60s.

In some embodiments, in addition to the data-sequence files, the system also has multiple labels to match the multiple experts. In particularly, for each training data, the system has multiple treatments (labels), and each label corresponds to the ideal output of an expert.

FIG. 10 is a flowchart depicting a method according to one aspect of the invention. At step 1000, the input data is received. The input data is then preprocessed at step 1010. The resulting preprocessed data is then analyzed with respect to a plurality of specific predefined medical areas/areas of expertise, at step 1020. Treatment options related to those specific predefined medical areas are generated at step 1030. As would be clear, there is no limit on the number of specific predefined medical areas/areas of expertise for which recommendations can be generated. The various options are then used to generate at least one final treatment suggestion, at step 1040. At step 1050, the at least one final treatment suggestion is presented to a user. It should be clear that, in one implementation of the present invention, step 1040 is executed by the expert manager. For this implementation, the various options from the various medical areas/areas of expertise are weighted accordingly to result in the one or more final treatment suggestion. Of course, this weighting process, as well as the process for determining which options are given more weight, how the one or more final treatment suggestion is arrived at, may be based on the known data about the patient as well as the configuration/training of the expert manager. As well, the one or more final treatment suggestion can be presented to the user, a person connected to the user, or to a person involved in the health/medical treatment of the user. The expert manager that dispenses the one or more final treatment suggestion may also be embodied in an autonomous entity such as a robot.

It should be clear that the various aspects of the present invention may be implemented as software modules in an overall software system. As such, the present invention may thus take the form of computer executable instructions that, when executed, implements various software modules with predefined functions.

Additionally, it should be clear that, unless otherwise specified, any references herein to ‘image’ or to ‘images’ refer to a digital image or to digital images, comprising pixels or picture cells. Likewise, any references to an ‘audio file’ or to ‘audio files’ refer to digital audio files, unless otherwise specified. ‘Video’, ‘video files’, ‘data objects’, ‘data files’ and all other such terms should be taken to mean digital files and/or data objects, unless otherwise specified.

The embodiments of the invention may be executed by a computer processor or similar device programmed in the manner of method steps, or may be executed by an electronic system which is provided with means for executing these steps. Similarly, an electronic memory means such as computer diskettes, CD-ROMs, Random Access Memory (RAM), Read Only Memory (ROM) or similar computer software storage media known in the art, may be programmed to execute such method steps. As well, electronic signals representing these method steps may also be transmitted via a communication network.

Embodiments of the invention may be implemented in any conventional computer programming language. For example, preferred embodiments may be implemented in a procedural programming language (e.g., “C” or “Go”) or an object-oriented language (e.g., “C++”, “java”, “PHP”, “PYTHON” or “C #”). Alternative embodiments of the invention may be implemented as pre-programmed hardware elements, other related components, or as a combination of hardware and software components.

Embodiments can be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink-wrapped software), preloaded with a computer system (e.g., on system ROM or fixed disk), or distributed from a server over a network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

A person understanding this invention may now conceive of alternative structures and embodiments or variations of the above all of which are intended to fall within the scope of the invention as defined in the claims that follow.

We claim:

1. A system for generating a recommendation for an individual, said system comprising:
a data-abstraction module for receiving input data related to said individual and for producing preprocessed data based on said input data; and
an expert module for analyzing said preprocessed data, said expert module comprising:
a plurality of expert units, wherein each of said plurality of expert units is configured to analyze said preprocessed data with respect to a specific predefined medical area, and each of said plurality of expert units is also configured to generate at least one treatment option related to said specific predefined medical area; and
an expert manager, wherein said expert manager is configured to receive outputs from said plurality of expert units and to generate at least one final treatment suggestion based on said outputs,
wherein said at least one final treatment suggestion is presented to at least one user.

2. The system of claim 1, wherein said at least one user is at least one of: said individual; a medical practitioner; or a caregiver for said individual.

3. The system of claim 1, wherein said input data comprises at least one of:
a medical image;
a medical record;
diet-related data;
activity-related data;
sleep-related data;
blood-pressure data;
preferences of said individual;
data related to a history of said individual;
data related to a family history of said individual;
health-related data for said individual;
text data;
image data;
video data;
medical imaging data;
numerical data;
unidimensional data; or
multi-dimensional data.

4. The system of claim 1, wherein said preprocessed data is in a form of a vector.

5. The system of claim 1, wherein said data-abstraction module uses at least one artificial-intelligence-based technique.

6. The system of claim 1, wherein said data-abstraction module comprises at least one of: at least one long-short term memory unit; or a convolutional neural network layer and a plurality of fully-connected layers.

7. The system of claim 1, wherein said expert manager uses at least one artificial-intelligence-based technique.

8. The system of claim 1, wherein each of said expert units is a machine-learning trained module.

9. The system of claim 1, wherein at least one of said input data and said preprocessed data is passed to said expert manager, and wherein said at least one final treatment suggestion is based on said at least one of said input data and said preprocessed data.

10. A method for generating a recommendation for an individual, said method comprising:
receiving input data related to said individual;
producing preprocessed data based on said input data;
analyzing said preprocessed data with respect to a plurality of specific predefined medical areas, to thereby generate at least one treatment option related to each of said plurality of specific predefined medical areas;
generating at least one final treatment suggestion based on outputs of said analyzing step; and
presenting said at least one final treatment suggestion to at least one user.

11. The method of claim 10, wherein said at least one user is one of: said individual; a medical practitioner; and a caregiver for said individual.

12. The method of claim 10, wherein said input data comprises at least one of:
a medical image;
a medical record;
diet-related data;
activity-related data;
sleep-related data;
blood-pressure data;
preferences of said individual;
data related to a history of said individual;
data related to a family history of said individual;
health-related data for said individual;
text data;
image data;
video data;
medical imaging data;
numerical data;
unidimensional data; or
multi-dimensional data.

13. The method of claim 10, wherein said preprocessed data is in a form of a vector.

14. The method of claim 10, wherein producing said preprocessed data uses at least one artificial-intelligence-based technique.

15. The method of claim 10, wherein at least one of: at least one long-short term memory unit; or a convolutional neural network layer and a plurality of fully-connected layers, are used in producing said preprocessed data.

16. The method of claim 10, wherein said analyzing is performed using a plurality of machine learning modules, wherein each of said machine learning modules has been trained to generate at least one treatment option related to said specific predefined medical area.

17. The method of claim 10, wherein a machine learning module is used in generating said at least one final treatment suggestion.

18. The method of claim 10, wherein said final treatment suggestion is based on said at least one of said input data and said preprocessed data.

19. Non-transitory computer-readable media having encoded thereon computer-readable and computer-executable instructions that, when executed, implement a method for generating a recommendation for an individual, said method comprising:
receiving input data related to said individual;
producing preprocessed data based on said input data;
analyzing said preprocessed data with respect to a plurality of specific predefined medical areas, to thereby generate at least one treatment option related to each of said plurality of specific predefined medical areas;
generating at least one final treatment suggestion based on said recommendations; and
presenting said at least one final treatment suggestion to at least one user.
